(19) United States
(12) Patent Application Publication
(10) Pub. No.: US 2025/0041500 A1
(43) Pub. Date: Feb. 6, 2025

(54) FLUSHING CONNECTION FOR AN EXTRACORPOREAL BLOOD TREATMENT DEVICE, CORRESPONDING BLOOD TREATMENT DEVICE AND METHOD FOR FLUSHING THE BLOOD TUBING SYSTEM OF THE BLOOD TREATMENT DEVICE

(71) Applicant: FRESENIUS MEDICAL CARE DEUTSCHLAND GMBH, Bad Homburg (DE)

(72) Inventor: Peter KLOFFEL, Nudlingen (DE)

(73) Assignee: FRESENIUS MEDICAL CARE DEUTSCHLAND GMBH, Bad Homburg (DE)

(21) Appl. No.: 18/718,805

(22) PCT Filed: Dec. 6, 2022
(86) PCT No.: PCT/EP2022/084635

§ 371 Date: Jun. 12, 2024

(30) Foreign Application Priority Data
Dec. 15, 2021 (DE) 10 2021 133 300.8

(51) Int. Cl. A61M 1/36

(52) U.S. Cl. CPC A61M 1/3649

(57) ABSTRACT

The invention relates to a flushing connection 2 for a medical treatment device, in particular an extracorporeal blood treatment device, for connecting a connector 14 of a fluid system 15 to be flushed, in particular an extracorporeal blood tubing system, for draining flushing liquid from the fluid system 15 to the medical treatment device. The inventive flushing connection 2 has a housing body 9 comprising a cavity 11 open on a front housing part 9A of the housing body 9 for receiving a flushing liquid, the housing body 9 having a connection part 13 at the front opening 12 embodied for connecting the connector 14 of the fluid system 15 to be flushed or a closure part 13 for liquid-tight closing of the opening 12 of the cavity 11, wherein the housing body 9 has a discharge connection piece 5 for connecting a discharge line 7 of the fluid system 1 of the medical treatment device, which is in fluid connection with the cavity 11. The flushing connection 2 is characterised in that a UV light emitting irradiation unit 21 is provided on the housing body 9 and is embodied such that the UV light irradiates at least part of the wall 11A of the cavity 11 of the housing body 9.

The invention relates to a flushing connection for a medical treatment device, in particular an extracorporeal blood treatment device, for connecting a connector of a fluid system to be flushed, in particular an extracorporeal blood tubing system, for draining flushing liquid from the fluid system to the medical treatment device. In addition, the invention relates to a medical treatment device, in particular an extracorporeal blood treatment device with a flushing connection, and to a method for flushing a blood tubing system of an extracorporeal blood treatment device, and to a method for operating an extracorporeal blood treatment device.

In medical technology, for removing or supplying a fluid, connection systems, also known as ports, are used that allow a sterile connection of the connector of a tubing line in order to be able to supply or remove a fluid. Connection systems of this type are generally located on a front side of the housing of medical devices in order to be easily accessible.

Haemodialysis devices are known which have a connection system to which a connector of a tubing line is connected in order to be able to supply substituate produced online in the dialysis machine to the extracorporeal blood circulation. When not in use, the connection system is tightly closed by a closure cap in order to prevent contamination. The closure cap is removed before the connector is connected. For the connection system, care must therefore be taken that germs and pathogens that can adhere to the connection system in daily practice do not get into the patient's blood. Therefore, the connection system is generally flushed with a disinfectant solution. After disinfection, no residues of the disinfectant solution should remain in order to reliably prevent the blood from coming into contact with the disinfectant solution.

EP 2 175 923 B1 discloses a UV irradiation unit having a hinged cylindrical housing in which a plurality of UV light-emitting diodes (LEDs) are integrated and are distributed around the circumference of the cylindrical housing. The irradiation unit should enclose the tubing coupling of a fluid line in order to be able to kill germs and pathogens.

U.S. Pat. No. 10,967,113 B2 describes a connection system for an extracorporeal blood treatment apparatus having a housing body which comprises an outer cylindrical housing part in which is formed a connection piece for a syringe for withdrawing a fluid. The connection system has a pivotable closure part for closing the housing opening. In the closure part, the base of which is made of a material that is permeable to ultraviolet light, there is a UV light-emitting diode that emits ultraviolet light. The UV light enters the cylindrical housing part of the housing body through the transparent base of the closure part and the housing opening in order to kill germs and pathogens.

To put an extracorporeal blood treatment device into operation, before the blood treatment is carried out an extracorporeal blood tubing system intended for single use (disposable), which comprises an arterial blood line with an arterial patient connection and a venous blood line with a venous patient connection, is flushed with a flushing liquid. During the flushing, the flushing liquid is pumped through the blood tubing system by means of the blood pump of the extracorporeal blood treatment device. The flushing liquid is discharged via a fluid line connected to a flushing connection provided on the extracorporeal blood treatment device.

The invention is based on the object of creating a flushing connection for a medical treatment device, in particular an extracorporeal blood treatment device, for connecting a connector of a fluid system to be flushed, in particular an extracorporeal blood tubing system, for draining flushing liquid from the fluid system to the medical treatment device, which reduces the risk of contamination with germs and pathogens. In addition, an object of the invention is to create a medical treatment device, in particular an extracorporeal blood treatment device, in which the risk of contamination with germs and pathogens during flushing of the fluid system is reduced. Another object of the invention is to provide a method for flushing an extracorporeal blood tubing system of an extracorporeal blood treatment device, in which method the risk of contamination with germs or pathogens is reduced during the flushing of the extracorporeal blood tubing system and to provide a method for operating an extracorporeal blood treatment device having a lower risk of contamination.

These objects are attained using the features of the independent claims. The dependent claims relate to advantageous embodiments of the invention.

The inventive flushing connection for a medical treatment device, in particular an extracorporeal blood treatment device, for connecting a connector of a fluid system to be flushed, in particular an extracorporeal blood tubing system, is intended for draining flushing liquid from the fluid system to the medical treatment device. The flushing connection has a housing body which comprises a cavity open on a front housing part of the housing body for receiving a flushing liquid, the housing body having a connection part at the front opening and being embodied for connecting the connector of the fluid system to be flushed or for connecting a closure part for liquid-tight closing of the opening of the cavity, wherein the housing body has a discharge connection piece for connecting a discharge line of the fluid system of the medical treatment device, which is in fluid communication with the cavity.

If the discharge line of the fluid system of the medical treatment device is connected to the discharge connection piece of the inventive flushing connection, flushing liquid can flow from the fluid system to be flushed through the cavity in the housing body to the discharge line during a discharge phase. In contrast, during the circulation phase, in which the flushing liquid circulates in the fluid system to be flushed, the flow of fluid from the fluid system to be flushed into the flushing connection is interrupted. In this case, the flushing liquid is in the cavity of the housing body, i.e. the flushing liquid does not flow through the cavity. In practice, it has been shown that germs and pathogens that may still be in the flushing connection can travel in the direction of the fluid system to be flushed, although the flushing liquid is in the cavity of the housing body. The reason for possible contamination is the pulsation of the pump pumping the flushing solution through the fluid system during the circulation phase.

The inventive flushing connection is characterised in that a UV light-emitting irradiation unit is provided on the housing body and is embodied such that the UV light irradiates at least part of the wall of the cavity of the housing body. The UV light irradiation unit is preferably arranged and embodied such that the UV light thereof essentially completely penetrates at least part of the cavity. The UV light kills germs and pathogens that could possibly still be in the flushing connection and could travel into the blood tubing system even though the flow of liquid from the fluid system to be flushed to the flushing connection is interrupted.

According to the inventive method, for flushing an extracorporeal blood tubing system of an extracorporeal blood treatment device the irradiation with UV light takes place during the circulation phase in which the flushing liquid is in the cavity, so that germs and pathogens that could travel into the blood tubing system to be flushed can be reliably killed even though the flow of liquid is interrupted. However, irradiation with UV light can also take place in the discharge phase.

After disinfection, the flushing connection for carrying out the extracorporeal blood treatment is closed in a liquid-tight manner with a closure part. According to the inventive method for operating an extracorporeal blood treatment device, the irradiation with UV light also takes place during the blood treatment, during which the cavity can contain liquid and/or air, so that the flushing connection is already prepared for the next application.

The UV light irradiation unit is not a component of the closure part for closing the opening of the connection piece during the blood treatment, but instead is located in or on the housing body of the flushing connection, i.e. on the inside of the machine if the flushing connection is arranged at the front of a medical device.

One embodiment of the inventive flushing connection provides that the UV light irradiation unit is arranged on a rear housing part of the housing body facing away from the front housing part. The arrangement of the irradiation unit on the rear of the housing enables the UV light to essentially completely penetrate the cavity and strike the wall thereof.

The UV light irradiation unit can have a UV LED. Instead of an LED, a plurality of UV LEDs, for example a UV LED array, can also be provided.

Another embodiment of the inventive flushing connection provides a UV light receiving unit, for receiving UV light, which is designed such that a control signal is generated when the UV light receiving unit receives UV light emitted by the UV light irradiation unit or a control signal is generated when the UV light receiving unit does not receive UV light emitted by the UV light irradiation unit. The UV light receiving unit allows the functionality of the UV light irradiation unit to be checked, which further increases safety.

The UV light receiving unit can be arranged in the region of the front opening of the housing body, so that the UV light receiving unit opposes the UV light irradiation unit when the irradiation unit is arranged on the rear of the housing. As a result, the UV light emitted by the irradiation unit can fall directly on the UV light receiving unit.

The housing body of the flushing connection can have further connection pieces in addition to the discharge connection piece. To connect an intake line, an intake connection piece opposing the discharge connection piece can be provided. The two connection pieces allow the flushing connection to be installed in a fluid line, it being possible for liquid to flow through the cavity of the housing body in one direction.

One exemplary embodiment of the invention is explained in more detail below with reference to the drawings.

### In the Figures:

FIG. 1 is a simplified schematic representation of a medical treatment system which has the inventive flushing connection;

FIG. 2 is a simplified schematic representation of the inventive flushing connection to which the fluid system to be flushed is connected; and,

FIG. 3 depicts the inventive flushing connection which is closed with a closure part.

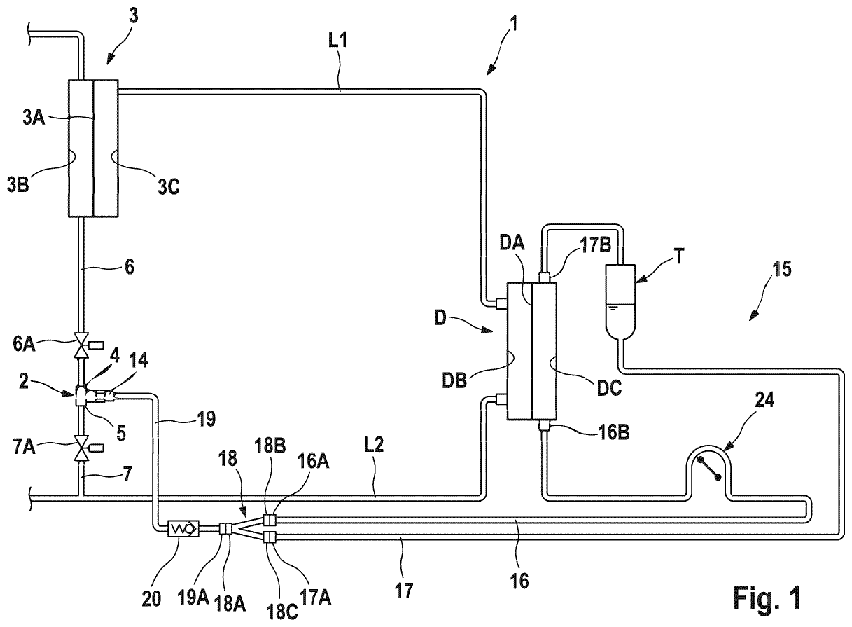

FIG. 1, in a greatly simplified schematic representation, depicts a medical treatment system during the flushing process and having the inventive flushing connection. In the present exemplary embodiment, the medical treatment system is a blood treatment system comprising an extracorporeal blood treatment device with an extracorporeal blood tubing system to be flushed. Only part of the fluid system of the extracorporeal blood treatment device is shown in FIG. 1.

The fluid system 1 of the extracorporeal blood treatment device comprises the inventive flushing connection 2 and a dialyser D, which is divided by a semipermeable membrane DA into the dialysis fluid chamber DB and a blood chamber DC, and a sterile filter 3, which is divided by a semipermeable membrane 3A into a first chamber 3B and a second chamber 3C. A dialysis fluid supply line L1 leads from an outlet of the second chamber 3C of the sterile filter 3 to an inlet of the dialysis fluid chamber DB and a dialysis fluid outflow line L2 leads from an outlet of the dialysis fluid chamber to a discharge element (not shown).

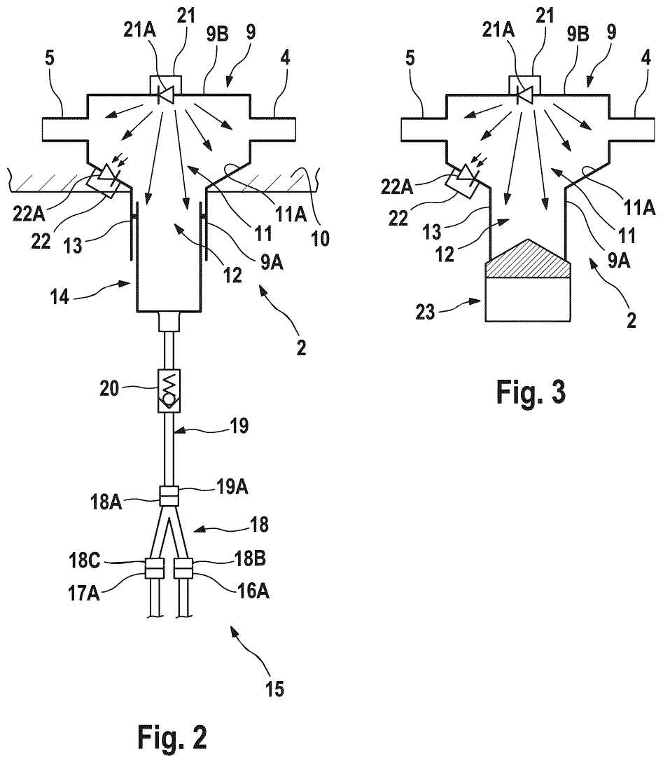

The flushing connection 2, which is described in detail below with reference to FIGS. 2 and 3, has an intake connection piece 4 and a discharge connection piece 5. One end of an intake line 6 is connected to the outlet of the first chamber 3A of the sterile filter 3, and the other end is connected to the intake connection piece 4 of the flushing connection 2. A discharge line 7 is connected to the discharge connection piece 5 of the flushing connection and leads to the dialysis fluid outflow line L2, which in turn leads via further fluid lines (not shown) to the discharge element (not shown) for draining flushing liquid. A shut-off element 6A is provided on the intake line 6 and a shut-off element 7A is provided on the discharge line 7, and these shut-off elements can be solenoid-operated shut-off valves.

FIGS. 2 and 3 depict the inventive flushing connection in greatly simplified schematic representations. The flushing connection 2 has a housing body 9 which has a front housing part 9A and a rear housing part 9B. The front and rear housing parts 9A, 9B can form a one-piece housing body or a housing body comprising several parts. The front housing part 9A is attached to a part of the housing 10 of a blood treatment device, in particular to a front panel, so that this housing part is easily accessible for the operating personnel. Embodied in the interior of the housing body 9 is a cavity 11 which is open on the front housing part 9A. The front housing part 9A is embodied in the region of the opening 12 of the cavity 11 as a connection part 13 for a connector 14 of an extracorporeal blood tubing system 15.

FIG. 2 shows the flushing connection 2, the connector 14 of the blood tubing system 15 being connected to the connection part 13 of the flushing connection 2. The intake connection piece 4 and the discharge connection piece 5, which are opposite one another on a common axis, are provided on the rear housing part 9B.

The extracorporeal blood tubing system 15 to be flushed comprises an arterial tubing line 16 with an arterial patient connection 16A at one end and a connection piece 16B at another end as well as a venous tubing line 17 with a venous patient connection 17A at one end and a connection piece 17B at another end. An arterial or venous cannula (not shown) can be connected to the patient connections 16A or 17A for the blood treatment. The connection piece 16B of the arterial blood line 16 is connected to an inlet of the blood chamber DC and the connection piece 17B of the venous blood line is connected to an outlet of the blood chamber DC of the dialyser D. For conveying fluids through the tubing lines, the extracorporeal blood treatment device has a pump 24, which can be a roller pump into which the arterial tubing line 16 can be inserted. A drip chamber T is connected to the venous blood line 17. In addition, the blood tubing system 15 to be flushed comprises a Y-tubing connection piece 18 with a first connection piece 18A and a second and third connection piece 18B and 18C, as well as a connection line 19 with the connector 14 at the one end and a connection piece 19A at the other end. A shut-off element 20 is provided on the connection line 19. In the present exemplary embodiment, the shut-off element 20 is a check valve which is arranged such that a flow of liquid from the blood tubing system 15 is only possible in the direction of the flushing connection 2, but not vice versa.

For flushing the blood tubing system 15, the connector 14 thereof is connected to the connection part 13 of the flushing connection 2, and the patient connection 16A or 17A of the arterial or venous blood line 16, 17 is connected to the second or third connection piece 18B or 18C of the Y-tubing connection piece 18 so that the flushing liquid can circulate in the tubing lines. The flushing liquid is conveyed by means of the pump 24 during this circulation phase. During the circulation phase, the fluid connection to the flushing connection 2 is interrupted, so that the fluid is in the cavity 11 of the housing body 9 of the flushing connection 2.

The used flushing liquid is drained off during a discharge phase, the flushing liquid flowing through the connection line 19 into the cavity 11 and out of the cavity through the discharge line 7 to the discharge element (FIG. 1). Fresh dialysis fluid can flow in via the semipermeable membrane of the dialyser.

The flushing connection 2 has a UV light irradiation unit 21 comprising one or more UV light emitting diodes (LEDs) 21A. The UV light irradiation unit 21, which is provided on the rear housing part 9B, irradiates the opposing connection part 13 and the wall 11A of the cavity 11 of the housing body 9 during the circulation phase, in which circulation phase the flushing liquid circulates in the blood tubing system 15, so that any germs or pathogens present in the flushing connection, which could otherwise travel into the blood tubing system 15 due to the pulsation of the pump 24, are reliably killed.

The used flushing liquid is drained during the discharge phase, the flushing liquid flowing through the connection line 19 into the cavity 11 of the housing body 9 and out of said cavity through the discharge line 7 to the discharge element (FIG. 1), the shut-off element 7 being open. During the discharge phase, the connection part 13 and the wall 11A of the cavity 11 of the housing body 9 can also be irradiated with UV light by means of the UV light irradiation unit 21 to kill germs and pathogens.

In addition, the flushing connection 2 has a UV light receiving unit 22 for receiving UV light which has a UV detector 22A, for example a photodiode, which in the present embodiment is provided on the front housing part 9A, so that the UV light can strike the photodiode. The UV light receiving unit 22 is embodied such that a control signal is generated when the receiving unit receives UV light emitted by the irradiation unit 21 or a control signal is generated when the receiving unit does not receive UV light emitted by the irradiation unit. This control signal can be evaluated in an evaluation unit (not shown) of the blood treatment device. For example, an alarm unit (not shown) can be used to generate an acoustic and/or visual alarm if the UV light receiving unit does not receive UV light emitted by the irradiation unit, so that operating personnel are prompted to take appropriate measures.

FIG. 3 depicts the flushing connection 2 during the actual extracorporeal blood treatment. During the blood treatment, the connection part 13 of the flushing connection is closed in a liquid-tight manner with a closure part 23 which is connected to the connection part 13 instead of the connector 14. During the blood treatment, the wall 11A of the cavity 11 and the connection part 13 are also irradiated with UV light by means of the UV light irradiation unit 21.

1. A flushing connection for a medical treatment device for connecting a connector of a fluid system to be flushed for draining flushing liquid from the fluid system to the medical treatment device, having a housing body comprising a cavity open on a front housing part of the housing body for receiving a flushing liquid, the housing body having a connection part at the front opening and embodied for connecting the connector of the fluid system to be flushed or for connecting a closure part for liquid-tight closing of the opening of the cavity, wherein the housing body has a discharge connection piece for connecting a discharge line of the fluid system of the medical treatment device which is in fluid communication with the cavity, wherein
a UV light irradiation unit for emitting UV light is provided on the housing body and is embodied such that the UV light irradiates at least part of a wall of the cavity of the housing body.

2. The flushing connection according to claim 1, wherein the UV light irradiation unit is arranged on a rear housing part of the housing body facing away from the front housing part.

3. The flushing connection according to claim 1, wherein the UV light irradiation unit has at least one UV LED.

4. The flushing connection according to claim 1, wherein a UV light receiving unit is provided for receiving UV light and is designed such that a control signal is generated when the UV light receiving unit receives UV light emitted by the UV light irradiation unit or a control signal is generated when the UV light receiving unit does not receive UV light emitted by the UV light irradiation unit.

5. The flushing connection according to claim 4, wherein the UV light receiving unit is arranged in the region of the front opening of the housing body.

6. The flushing connection according to claim 1, wherein the housing body has an intake connection piece opposite the discharge connection piece for connecting an intake line and is in fluid communication with the cavity.

7. A medical treatment device which has a fluid system which has an outlet line for draining a flushing liquid, wherein the medical treatment device has a flushing connection according to claim 1, wherein the outlet line of the fluid system of the medical treatment device is connected to the outlet connection piece of the flushing connection.

8. The medical treatment device according to claim 7, wherein a shut-off element is provided on the outlet line downstream of the flushing connection.

9. An extracorporeal blood treatment system having an extracorporeal blood treatment device which has a fluid system which has a discharge line for draining a flushing liquid, and
a flushing connection according to claim 1, wherein the discharge line of the fluid system of the extracorporeal blood treatment device is connected to the discharge connection piece of the flushing connection, and
an extracorporeal blood tubing system to be flushed, which has an arterial blood line with an arterial patient connection and a venous blood line with a venous patient connection, wherein the arterial and venous blood lines are connected by means of a Y-tube connection piece to which the one end of a connection line and at the other end of which a connector is connected which can be connected to the connection part of the flushing connection, wherein a shut-off element is arranged on the connection line.

10. A method for flushing an extracorporeal blood tubing system of an extracorporeal blood treatment device, the blood treatment device having a flushing connection which has a housing body comprising a cavity open on a housing part of the housing body for receiving a flushing liquid, the housing body having at the opening of the cavity a connection part to which a connector in fluid communication with the blood tubing system to be flushed is connected, wherein the housing body has an discharge connection piece which is in fluid communication with the cavity and to which a discharge line of the fluid system of the extracorporeal blood treatment device is connected, wherein during a circulation phase flushing liquid circulates in the blood tubing system to be flushed and the flow of liquid from the blood tubing system to be flushed into the flushing connection is interrupted, so that the fluid is in the cavity of the flushing connection and, during a discharge phase, flushing liquid flows from the blood tubing system to be flushed through the cavity to the discharge line, wherein
during the circulation phase, at least part of a wall of the cavity of the housing body of the flushing connection is irradiated with UV light.

11. The method according to claim 10, wherein during the discharge phase at least part of the wall of the cavity of the housing body of the flushing connection is irradiated with UV light.

12. The method according to claim 10, wherein the irradiation with UV light takes place by means of a UV light irradiation unit, the UV light of which essentially completely penetrates the cavity.

13. A method for operating an extracorporeal blood treatment device, the blood treatment device having a flushing connection which has a housing body comprising a cavity open on a housing part of the housing body for receiving a flushing liquid, wherein the housing body has a connection part at the opening of the cavity to which a connector that is in fluid communication with an extracorporeal blood tubing system of the extracorporeal blood treatment device to be flushed can be connected, wherein the housing body has a discharge connection piece that is in fluid communication with the cavity and to which a discharge line of the fluid system of the extracorporeal blood treatment device is connected, so that during a discharge phase, when the connector in fluid communication with the fluid system to be flushed is connected to the connection part, flushing liquid from the fluid system to be flushed can flow through the cavity to the discharge line, and wherein when the blood treatment is being carried out the opening of the housing body is closed in a liquid-tight manner with a closure part,
wherein while the blood treatment is being carried out at least part of the cavity is irradiated with UV light.

14. The method according to claim 13, wherein the irradiation with UV light takes place by means of a UV light irradiation unit, the UV light of which penetrates the cavity essentially completely.

15. The method according to claim 13, wherein a shut-off element is provided in the discharge line of the fluid system of the extracorporeal blood treatment device downstream of the flushing connection and is closed while the blood treatment is being carried out.

16. The flushing connection of claim 1, wherein said medical treatment device is an extracorporeal blood treatment device and the fluid system an extracorporeal blood tubing system.

17. The extracorporeal blood treatment system of claim 9, wherein the shut-off element is a check valve.
